SEQUENCE LISTING (41 sequences)

SEQ ID NO: 1 - protein, 118 aa, synthetic construct
QVQLVESGGGVVQPGRSLRLSCAASGFTFGSYGMHWVRQAPGKGLEWVATISYDGSIKDYADSVKGRFTISRDNSKNTLYLQMNSLRAEDTAVYYCAKGDRTGNLDYWGQGTLVTVSS

SEQ ID NO: 2 - protein, 127 aa, synthetic construct
QVQLVQSGAEVKKPGSSVKVSCKASGGTYSRYTLNWVRQAPGQGLEWMGGIIPIFGTANYAQRFQGRLTITADESTSTAYMELSSLRSDDTAVYFCARENLDNSGTYYYFSGWFDPWGQGTLVTVSS

SEQ ID NO: 3 - protein, 107 aa, synthetic construct
DIQMTQSPSSLSASVGDRVTITCRASQNIRNALNWYQQKPGKAPKLLIYDASTRQSGVPSRFSGSGSGTDFTLTISSLQPEDFATYYCQQNSEFPPTFGQGTKVEIK

SEQ ID NO: 4 - protein, 112 aa, synthetic construct
QSALTQPRSVSGSPGQSVTISCTGTSSDIGGYNYVSWYQQHPGKAPKLMIYDATKRPSGVPDRFSGSKSGNTASLTISGLQAEDEADYYCCSYAGDYTPGVVFGGGTKLTVL

SEQ ID NO: 5 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 107 aa, Homo sapiens [sequence omitted]

SEQ ID NO: 7 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 240 aa, synthetic construct
QVQLVESGGGVVQPGRSLRLSCAASGFTFGSYGMHWVRQAPGKGLEWVATISYDGSIKDYADSVKGRFTISRDNSKNTLYLQMNSLRAEDTAVYYCAKGDRTGNLDYWGQGTLVTVSSGGGGSGGGGSGGGGSDIQMTQSPSSLSASVGDRVTITCRASQNIRNALNWYQQKPGKAPKLLIYDASTRQSGVPSRFSGSGSGTDFTLTISSLQPEDFATYYCQQNSEFPPTFGQGTKVEIK

SEQ ID NO: 9 - protein, 254 aa, synthetic construct
QVQLVQSGAEVKKPGSSVKVSCKASGGTYSRYTLNWVRQAPGQGLEWMGGIIPIFGTANYAQRFQGRLTITADESTSTAYMELSSLRSDDTAVYFCARENLDNSGTYYYFSGWFDPWGQGTLVTVSSGGGGSGGGGSGGGGSQSALTQPRSVSGSPGQSVTISCTGTSSDIGGYNYVSWYQQHPGKAPKLMIYDATKRPSGVPDRFSGSKSGNTASLTISGLQAEDEADYYCCSYAGDYTPGVVFGGGTKLTVL

SEQ ID NO: 10 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - protein, 127 aa, synthetic construct
QVQLVQSGAEVKKPGSSVKVSCKASGGTFNRYTVNWVRQAPGQGLEWMGGIIPIFGTANYAQRFQGRLTITADESTSTAYMELSSLRSDDTAVYFCARENLDNSGTYYYFSGWFDPWGQGTLVTVSS

SEQ ID NO: 13 - protein, 119 aa, synthetic construct
EVQLVQSGGGAVQPGRSLRLSCAASGFTFSSYGMHWVRQAPGKGLEWVAVILYDGSDKFYADSVKGRFTISRDNSKNTLYLQMNSLRAEDTAVYYCAKVAVAGTHFDYWGQGTLVTVSS

SEQ ID NO: 14 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - protein, 112 aa, synthetic construct
QSALTQPRSVSGSPGQSVTISCTGTSSDIGGYNFVSWYQQHPGKAPKLMIYDATKRPSGVPDRFSGSKSGNTASLTISGLQAEDEADYYCCSYAGDYTPGVVFGGGTKLTVL

SEQ ID NO: 16 - protein, 107 aa, synthetic construct
DIQMTQSPSSLSASVGDRVTITCRASQGIRNDLGWYQQKPGKAPKLLIYAASSLQSGVPSRFSGSGSGTDFTLTISSLQPEDFATYYCQQLNSYPPTFGGGTKVEIK

SEQ ID NO: 17 - protein, 106 aa, Mus musculus
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 18 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 254 aa, synthetic construct
QVQLVQSGAEVKKPGSSVKVSCKASGGTYSRYTINWVRQAPGQGLEWMGGIIPIFGTANYAQRFQGRLTITADESTSTAYMELSSLRSDDTAVYFCARENLDNSGTYYYFSGWFDPWGQGTLVTVSSGGGGSGGGGSGGGGSQSALTQPRSVSGSPGQSVTISCTGTSSDIGAYDYVSWYQQHPGKAPKLMIYDATKRPSGVPDRFSGSKSGNTASLTISGLQAEDEADYYCCSYAGDYTPGVVFGGGTKLTVL

SEQ ID NO: 22 - protein, 254 aa, synthetic construct
QVQLVQSGAEVKKPGSSVKVSCKASGGTYSRYSINWVRQAPGQGLEWMGGIIPIFGTANYAQRFQGRLTITADESTSTAYMELSSLRSDDTAVYFCARENLDNSGTYYYFSGWFDPWGQGTLVTVSSGGGGSGGGGSGGGGSQSALTQPRSVSGSPGQSVTISCTGTSSDIDGYDFVSWYQQHPGKAPKLMIYDATKRPSGVPDRFSGSKSGNTASLTISGLQAEDEADYYCCSYAGDYTPGVVFGGGTKLTVL

SEQ ID NO: 23 - protein, 254 aa, synthetic construct
QVQLVQSGAEVKKPGSSVKVSCKASGGTYSGYTINWVRQAPGQGLEWMGGIIPIFGTANYAQRFQGRLTITADESTSTAYMELSSLRSDDTAVYFCARENLDNSGTYYYFSGWFDPWGQGTLVTVSSGGGGSGGGGSGGGGSQSALTQPRSVSGSPGQSVTISCTGTSSDLGGYDFVSWYQQHPGKAPKLMIYDATKRPSGVPDRFSGSKSGNTASLTISGLQAEDEADYYCCSYAGDYTPGVVFGGGTKLTVL

SEQ ID NO: 24 - protein, 127 aa, synthetic construct
QVQLVQSGAEVKKPGSSVKVSCKASGGTYSRYTINWVRQAPGQGLEWMGGIIPIFGTANYAQRFQGRLTITADESTSTAYMELSSLRSDDTAVYFCARENLDNSGTYYYFSGWFDPWGQGTLVTVSS

SEQ ID NO: 25 - protein, 112 aa, synthetic construct
QSALTQPRSVSGSPGQSVTISCTGTSSDIGAYDYVSWYQQHPGKAPKLMIYDATKRPSGVPDRFSGSKSGNTASLTISGLQAEDEADYYCCSYAGDYTPGVVFGGGTKLTVL

SEQ ID NO: 26 - protein, 127 aa, synthetic construct
QVQLVQSGAEVKKPGSSVKVSCKASGGTYSRYSINWVRQAPGQGLEWMGGIIPIFGTANYAQRFQGRLTITADESTSTAYMELSSLRSDDTAVYFCARENLDNSGTYYYFSGWFDPWGQGTLVTVSS

SEQ ID NO: 27 - protein, 112 aa, synthetic construct
QSALTQPRSVSGSPGQSVTISCTGTSSDIDGYDFVSWYQQHPGKAPKLMIYDATKRPSGVPDRFSGSKSGNTASLTISGLQAEDEADYYCCSYAGDYTPGVVFGGGTKLTVL

SEQ ID NO: 28 - protein, 127 aa, synthetic construct
QVQLVQSGAEVKKPGSSVKVSCKASGGTYSGYTINWVRQAPGQGLEWMGGIIPIFGTANYAQRFQGRLTITADESTSTAYMELSSLRSDDTAVYFCARENLDNSGTYYYFSGWFDPWGQGTLVTVSS

SEQ ID NO: 29 - protein, 112 aa, synthetic construct
QSALTQPRSVSGSPGQSVTISCTGTSSDLGGYDFVSWYQQHPGKAPKLMIYDATKRPSGVPDRFSGSKSGNTASLTISGLQAEDEADYYCCSYAGDYTPGVVFGGGTKLTVL

SEQ ID NO: 30 - protein, 214 aa, Homo sapiens
DIQMTQSPSSLSASVGDRVTITCRASQNIRNALNWYQQKPGKAPKLLIYDASTRQSGVPSRFSGSGSGTDFTLTISSLQPEDFATYYCQQNSEFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 448 aa, synthetic construct
QVQLVESGGGVVQPGRSLRLSCAASGFTFGSYGMHWVRQAPGKGLEWVATISYDGSIKDYADSVKGRFTISRDNSKNTLYLQMNSLRAEDTAVYYCAKGDRTGNLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 488 aa, synthetic construct
QVQLVQSGAEVKKPGSSVKVSCKASGGTYSGYTINWVRQAPGQGLEWMGGIIPIFGTANYAQRFQGRLTITADESTSTAYMELSSLRSDDTAVYFCARENLDNSGTYYYFSGWFDPWGQGTLVTVSSGGGGSGGGGSGGGGSQSALTQPRSVSGSPGQSVTISCTGTSSDLGGYDFVSWYQQHPGKAPKLMIYDATKRPSGVPDRFSGSKSGNTASLTISGLQAEDEADYYCCSYAGDYTPGVVFGGGTKLTVLASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 488 aa, synthetic construct
QVQLVQSGAEVKKPGSSVKVSCKASGGTYSRYTINWVRQAPGQGLEWMGGIIPIFGTANYAQRFQGRLTITADESTSTAYMELSSLRSDDTAVYFCARENLDNSGTYYYFSGWFDPWGQGTLVTVSSGGGGSGGGGSGGGGSQSALTQPRSVSGSPGQSVTISCTGTSSDIGAYDYVSWYQQHPGKAPKLMIYDATKRPSGVPDRFSGSKSGNTASLTISGLQAEDEADYYCCSYAGDYTPGVVFGGGTKLTVLASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 488 aa, synthetic construct
QVQLVQSGAEVKKPGSSVKVSCKASGGTYSRYSINWVRQAPGQGLEWMGGIIPIFGTANYAQRFQGRLTITADESTSTAYMELSSLRSDDTAVYFCARENLDNSGTYYYFSGWFDPWGQGTLVTVSSGGGGSGGGGSGGGGSQSALTQPRSVSGSPGQSVTISCTGTSSDIDGYDFVSWYQQHPGKAPKLMIYDATKRPSGVPDRFSGSKSGNTASLTISGLQAEDEADYYCCSYAGDYTPGVVFGGGTKLTVLASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 440 aa, Rabies virus
KFPIYTIPDKLGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKY

SEQ ID NO: 36 - protein, 440 aa, synthetic construct
KFPIYTIPDKLGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASKGNKTCGFVDERGLYKSLKGACRLKLCEVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKY

SEQ ID NO: 37 - protein, 440 aa, synthetic construct
KFPIYTIPDKLGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNETIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKY

SEQ ID NO: 38 - protein, 32 aa, Homo sapiens
VSRLEEEMRKLQATVQELQKRLDRLEETVQAK

SEQ ID NO: 39 - protein, 488 aa, synthetic construct
KFPIYTIPDKLGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKYGGGGSVSRLEEEMRKLQATVQELQKRLDRLEETVQAKASGAAHHHHHH

SEQ ID NO: 40 - protein, 488 aa, synthetic construct
KFPIYTIPDKLGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASKGNKTCGFVDERGLYKSLKGACRLKLCEVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKYGGGGSVSRLEEEMRKLQATVQELQKRLDRLEETVQAKASGAAHHHHHH

SEQ ID NO: 41 - protein, 488 aa, synthetic construct
KFPIYTIPDKLGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNETIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKYGGGGSVSRLEEEMRKLQATVQELQKRLDRLEETVQAKASGAAHHHHHH

(19) United States
(12) Patent Application Publication
(10) Pub. No.: US 2025/0019421 A1
(43) Pub. Date: Jan. 16, 2025

(54) PHARMACEUTICAL FORMULATION COMPRISING BISPECIFIC ANTIBODY AGAINST RABIES VIRUS G PROTEIN AND PREPARATION METHOD THEREFOR

(71) Applicants: CHONGQING GENRIX BIOPHARMACEUTICAL CO., LTD., Chongqing (CN); GENRIX (SHANGHAI) BIOPHARMACEUTICAL CO.LTD., Shanghai (CN)

(72) Inventors: Zhiyuan CHANG, Chongqing (CN); Zhitong ZOU, Chongqing (CN); Xin ZHANG, Chongqing (CN); Wei WANG, Chongqing (CN)

(21) Appl. No.: 18/851,891

(22) PCT Filed: Apr. 3, 2023
(86) PCT No.: PCT/CN2023/085933

§ 371 Date: Sep. 27, 2024

(30) Foreign Application Priority Data
Apr. 2, 2022 (CN) 202210357290.X

(51) Int. Cl. C07K 16/10; A61K 47/18; A61K 47/26

(52) U.S. Cl. CPC C07K 16/10; A61K 47/183; A61K 47/26; C07K 2317/31; C07K 2317/76; C07K 2317/94

(57) ABSTRACT

The present application provides a pharmaceutical formulation, such as a liquid formulation, comprising a bispecific antibody against rabies virus G protein and an antioxidant. The bispecific antibody comprises two antigen-binding fragments respectively binding to epitope I and epitope III of the rabies virus G protein, and has the activity of neutralizing rabies virus. The formulation can be used in post-exposure prophylaxis of suspected rabies virus, and is administered by injection.

### CROSS-REFERENCE TO RELATED APPLICATION

The present application claims the benefit and priority to the Chinese Patent Application No. 202210357290.X filed before the China National Intellectual Property Administration on Apr. 2, 2022, the content of which is incorporated herein by reference in its entirety.

### TECHNICAL FIELD

The present application generally relates to the field of a pharmaceutical formulation comprising an antibody. Specifically, the present application relates to a stable pharmaceutical formulation comprising a bispecific antibody against rabies virus G protein and a preparation method thereof.

### BACKGROUND

Rabies virus is a bullet-shaped, enveloped, single-stranded RNA virus, and belongs to the Rhabdoviridae family, and the lyssavirus genus. The genome of rabies virus encodes five viral proteins, i.e. RNA-dependent RNA aggregatesase (L), nucleoprotein (N), phosphorylated protein (P), matrix protein (M) located inside the envelope of the viral protein, and outer surface glycoprotein (G). The glycoprotein (G protein) of rabies virus binds to acetylcholine, which determines the neurophagocytosis of rabies virus. The G protein (62-67 kDa) is a type I glycoprotein consisting of 505 amino acids, forms a protuberance covering the outer surface of the viral particle envelope, and has been shown to induce viral neutralizing antibodies. The G protein has at least five neutralizing epitopes. Epitope II is a discontinuous spatial epitope including amino acid residues 34-42 and amino acid residues 198-200. Epitope III is located at positions 330-338 and is a linear epitope. About 97% of the reported antibodies recognize epitope II and epitope III. Rabies virus neutralizing antibody CR4098 binds to epitope III. Few antibodies recognize epitope I and epitope IV. Rabies virus neutralizing antibody CR57 recognizes linear epitope I, i.e., position 218-240, in which the core binding domain is KLCGVL at position 226-231. Epitope IV contains residues 251 and 264. Yet another epitope is microepitope a that does not overlap with epitope III and separate from epitope III by three amino acid residues, and has only two amino acid residues 342-343.

For prevention and treatment of rabies virus, WHO recommends that, for class III exposure and exposure above class II with wildlife bites, the subjects should receive both active and passive immunotherapies for rapid protection. Currently, a bispecific antibody against rabies virus G protein has been developed for passive immunotherapy.

Like any protein, the biological activity of an antibody depends upon the conformational integrity of at least a core sequence of amino acids remaining intact while protecting the protein's multiple functional groups from degradation. Chemical and physical instability can each contribute to degradation of an antibody. Because antibodies are larger and more complex than traditional organic and inorganic drugs, the formulation of such antibodies poses special problems. Antibody stability can be affected by various factors, such as pH, temperature, repeated cycles of freeze/thaw, and shear forces. Active antibodies may be lost as a result of physical instabilities, including denaturation, aggregation (both soluble and subvisible aggregate formation), precipitation and adsorption, as well as chemical instabilities, including, for example, racemization, hydrolysis, and deamidation, and so on. Any of these instabilities can potentially result in the formation of antibody by-products or derivatives having lowered biological activity, increased toxicity, and/or increased immunogenicity.

While the prior art indicates numerous examples of excipients that can be suitably employed to create antibody formulations for specific antibodies, it is impossible to predict which excipients should be added and in what amount they should be added to overcome the particular instability problems that a particular antibody may have. Furthermore, it is difficult to find optimal conditions that keep a particular antibody chemically and biologically stable within a particular formulation. In view of all the factors that can be varied, finding suitable excipients and optimal conditions for formulating an antibody is fraught with challenges.

Therefore, there is still a need in the art for a stable pharmaceutical formulation comprising an antibody against rabies virus G protein that can be stored for a long period of time.

### SUMMARY OF THE INVENTION

In one aspect, the present application provides a pharmaceutical composition comprising about 0.2 mg/ml to about 20 mg/ml of a bispecific antibody against rabies virus G protein (hereinafter referred to as “bispecific antibody”), a tonicity modifier selected from trehalose and mannitol, a metal ion chelator type antioxidant, a nonionic surfactant and a balance of water, wherein the pharmaceutical composition has a pH of about 5.0 to about 6.2, and wherein the bispecific antibody comprises an antigen-binding fragment that binds to epitope I of the rabies virus G protein and an antigen-binding fragment that binds to epitope III of the rabies virus G protein, and the bispecific antibody has the activity of neutralizing rabies virus.

In one aspect, the present application provides a pharmaceutical composition comprising about 0.2 mg/ml to about 20 mg/ml of a bispecific antibody against rabies virus G protein (hereinafter referred to as “bispecific antibody”), a buffer, a tonicity modifier selected from trehalose and mannitol, a metal ion chelator type antioxidant, a nonionic surfactant and a balance of water, wherein the pharmaceutical composition has a pH of about 5.0 to about 6.2, and wherein the bispecific antibody comprises an antigen-binding fragment that binds to epitope I of the rabies virus G protein and an antigen-binding fragment that binds to epitope III of the rabies virus G protein, and the bispecific antibody has the activity of neutralizing rabies virus.

In some embodiments, the present application provides a pharmaceutical composition comprising about 0.2 mg/ml to about 20 mg/ml of the bispecific antibody, about 10 mM to about 50 mM of the buffer, about 100 mM to about 400 mM of the tonicity modifier selected from trehalose and mannitol, about 0.2 mM to about 5 mM of the metal ion chelator type antioxidant, about 0.05 mg/ml to about 1.0 mg/ml of the nonionic surfactant and a balance of water, wherein the pharmaceutical composition has a pH of about 5.0 to about 6.2.

In another aspect, the present application provides a use of a pharmaceutical composition in the preparation of a medicament for preventing or treating rabies, wherein the pharmaceutical composition comprises about 0.2 mg/ml to about 20 mg/ml of the bispecific antibody, a tonicity modifier selected from trehalose and mannitol, a metal ion chelator type antioxidant, a nonionic surfactant and a balance of water, wherein the pharmaceutical composition has a pH of about 5.0 to about 6.2.

In another aspect, the present application provides a method for preventing or treating rabies, comprising administering to a subject in need thereof a pharmaceutical composition comprising about 0.2 mg/ml to about 20 mg/ml of the bispecific antibodies, a tonicity modifier selected from trehalose and mannitol, a metal ion chelator type antioxidant, a nonionic surfactant and a balance of water, wherein the pharmaceutical composition has a pH of about 5.0 to about 6.2.

In another aspect, the present application provides a pharmaceutical composition for use in preventing or treating rabies, which comprises about 0.2 mg/ml to about 20 mg/ml of the bispecific antibody, a tonicity modifier selected from trehalose and mannitol, a metal ion chelator type antioxidant, a nonionic surfactant and a balance of water, wherein the pharmaceutical composition has a pH of about 5.0 to about 6.2.

In one embodiment of the present application, the pharmaceutical composition further comprises a buffer, such as about 10 mM to about 50 mM of the buffer.

In one embodiment of the present application, the metal ion chelator type antioxidant is ethylenediamine tetraacetate salt.

In some embodiments, the pharmaceutical composition of the present application can prevent or treat rabies by implementing passive immunization following suspected rabies virus exposure.

### BRIEF DESCRIPTION OF THE DRAWINGS

FIGS. 1A-1F illustrate the effect of tonicity modifiers on the stability of pharmaceutical formulations comprising the S2E3-SCFV-FCH+C34M-IGG1K antibody.

FIGS. 2A-2F illustrate the effect of antioxidants on the stability of pharmaceutical formulations comprising low concentrations of the S2E3-SCFV-FCH+C34M-IGG1K antibody.

FIGS. 3A-3F illustrate the effect of antioxidants on the stability of pharmaceutical formulations comprising high concentrations of the S2E3-SCFV-FCH+C34M-IGG1K antibody.

FIGS. 4A-4F illustrate the effect of pH values on the stability of pharmaceutical formulations comprising the S2E3-SCFV-FCH+C34M-IGG1K antibody in the absence of antioxidants.

FIGS. 5A-5F illustrate the effect of pH values on the stability of pharmaceutical formulations comprising the S2E3-SCFV-FCH+C34M-IGG1K antibody in the presence of antioxidants.

FIGS. 6A-6F illustrate the effect of buffers on the stability of pharmaceutical formulations comprising the S2E3-SCFV-FCH+C34M-IGG1K antibody.

FIGS. 7A-7E illustrate the effect of the concentrations of polysorbate 80 on the amount of subvisible particles and the level of SEC aggregates in pharmaceutical formulations comprising the S2E3-SCFV-FCH+C34M-IGG1K antibody in a shaking experiment.

FIGS. 8A-8F illustrate the effect of the presence of polysorbate 80 on the stability of pharmaceutical formulations comprising the S2E3-SCFV-FCH+C34M-IGG1K antibody.

FIGS. 9A-9F illustrate the effect of the concentrations of the S2E3-SCFV-FCH+C34M-IGG1K antibody on the stability of pharmaceutical formulations comprising the S2E3-SCFV-FCH+C34M-IGG1K antibody in an accelerated stability experiment.

FIGS. 10A-10F illustrate the effect of the concentrations of the S2E3-SCFV-FCH+C34M-IGG1K antibody on the stability of pharmaceutical formulations comprising the S2E3-SCFV-FCH+C34M-IGG1K antibody in a long-term stability experiment.

### DETAILED DESCRIPTION OF THE INVENTION

### Bispecific Antibody Against Rabies Virus G Protein

The bispecific antibody against rabies virus G protein of the present application comprises two antigen-binding fragments that bind to different epitopes of the rabies virus G protein, particularly an antigen-binding fragment that binds to epitope I of the rabies virus G protein and an antigen-binding fragment that binds to epitope III of the rabies virus G protein, and the bispecific antibody has the activity of neutralizing rabies virus.

The structure, amino acid sequence, preparation method and biological activity of the bispecific antibody against rabies virus G protein of the present application have been described in Chinese Patent Application No. 201910706710.9, the content of which is incorporated herein by reference in its entirety.

In some embodiments, the antigen-binding fragment that binds to epitope I of the rabies virus G protein comprises:
HCDR1 having the amino acid sequence of RYTIN, HCDR2 having the amino acid sequence of GIIPIFGTANYAQRFQG, HCDR3 having the amino acid sequence of ENLDNSGTYYYYFSGWFDP, LCDR1 having the amino acid sequence of TGTSSDIGAYDYVS, LCDR2 having the amino acid sequence of DATKRPS, and LCDR3 having the amino acid sequence of CSYAGDYTPGVV; or
HCDR1 having the amino acid sequence of RYSIN, HCDR2 having the amino acid sequence of GIIPIFGTANYAQRFQG, HCDR3 having the amino acid sequence of ENLDNSGTYYYYFSGWFDP, LCDR1 having the amino acid sequence of TGTSSDIDGYDFVS, LCDR2 having the amino acid sequence of DATKRPS, and LCDR3 having the amino acid sequence of CSYAGDYTPGVV; or
HCDR1 having the amino acid sequence of GYTIN, HCDR2 having the amino acid sequence of GIIPIFGTANYAQRFQG, HCDR3 having the amino acid sequence of ENLDNSGTYYYYFSGWFDP, LCDR1 having the amino acid sequence of TGTSSDLGGYDFVS, LCDR2 having the amino acid sequence of DATKRPS, and LCDR3 having the amino acid sequence of CSYAGDYTPGVV;
wherein the HCDR and LCDR amino acid sequences are defined according to Kabat.

In some embodiments, the antigen-binding fragment that binds to epitope III of the rabies virus G protein comprises:
HCDR1 having the amino acid sequence of SYGMH, HCDR2 having the amino acid sequence of TISYDGSIKDYADSVKG, HCDR3 having the amino acid sequence of GDRTGNLDY, LCDR1 having the amino acid sequence of RASQNIRNALN, LCDR2 having the amino acid sequence of DASTRQS, and LCDR3 having the amino acid sequence of QQNSEFPPT;
wherein the HCDR and LCDR amino acid sequences are defined according to Kabat.

In some embodiments, the amino acid sequence of the heavy chain variable region of the antigen-binding fragment that binds to epitope I of the rabies virus G protein is as set forth in SEQ ID NO: 24, and the amino acid sequence of the light chain variable region is as set forth in SEQ ID NO: 25; or
the amino acid sequence of the heavy chain variable region of the antigen-binding fragment that binds to epitope I of the rabies virus G protein is as shown in SEQ ID NO: 26, and the amino acid sequence of the light chain variable region is as shown in SEQ ID NO: 27; or
the amino acid sequence of the heavy chain variable region of the antigen-binding fragment that binds to epitope I of the rabies virus G protein is shown in SEQ ID NO: 28, and the amino acid sequence of the light chain variable region is shown in SEQ ID NO: 29.

In some embodiments, the amino acid sequence of the heavy chain variable region of the antigen-binding fragment that binds to epitope III of the rabies virus G protein is as set forth in SEQ ID NO: 1 and the amino acid sequence of the light chain variable region is as set forth in SEQ ID NO: 3.

In some embodiments, the amino acid sequence of the heavy chain variable region of the antigen-binding fragment that binds to epitope I of the rabies virus G protein is as set forth in SEQ ID NO: 24, and the amino acid sequence of the light chain variable region is as set forth in SEQ ID NO: 25; and the amino acid sequence of the heavy chain variable region of the antigen-binding fragment that binds to epitope III of the rabies virus G protein is as set forth in SEQ ID NO: 1, and the amino acid sequence of the light chain variable region is as shown in SEQ ID NO: 3; or
the amino acid sequence of the heavy chain variable region of the antigen-binding fragment that binds to epitope I of the rabies virus G protein is shown in SEQ ID NO: 26, and the amino acid sequence of the light chain variable region is shown in SEQ ID NO: 27; and the amino acid sequence of the heavy chain variable region of the antigen-binding fragment that binds to epitope III of the rabies virus G protein is shown in SEQ ID NO: 1, and the amino acid sequence of the light chain variable region is as shown in SEQ ID NO: 3; or
the amino acid sequence of the heavy chain variable region of the antigen-binding fragment that binds to epitope I of the rabies virus G protein is shown in SEQ ID NO: 28, and the amino acid sequence of the light chain variable region is shown in SEQ ID NO: 29; and the amino acid sequence of the heavy chain variable region of the antigen-binding fragment that binds to epitope III of the rabies virus G protein is shown in SEQ ID NO: 1, and the amino acid sequence of the light chain variable region is shown in SEQ ID NO: 3.

In some embodiments, the forms of the two antigen-binding fragments are independently selected from a single chain antibody (scFv) or a Fab fragment.

In some embodiments, the antigen-binding fragment that binds to epitope I of the rabies virus G protein is a single chain antibody (scFv) and the antigen-binding fragment that binds to epitope III of the rabies virus G protein is a Fab fragment. In some embodiments, the bispecific antibody comprises the amino acid sequence set forth in one of SEQ ID NOS: 32, 33, and 34. In some embodiments, the bispecific antibody comprises the amino acid sequences set forth in SEQ ID NO: 30 and SEQ ID NO: 31.

### Pharmaceutical Composition

In one aspect, the present application provides a pharmaceutical composition comprising about 0.2 mg/ml to about 20 mg/ml of the bispecific antibody, a tonicity modifier selected from trehalose and mannitol, a metal ion chelator type antioxidant, a nonionic surfactant and a balance of water, wherein the pharmaceutical composition has a pH of about 5.0 to about 6.2.

In one aspect, the present application provides a pharmaceutical composition comprising about 0.2 mg/ml to about 20 mg/ml of the bispecific antibody, a buffer, a tonicity modifier selected from trehalose and mannitol, a metal ion chelator type antioxidant, a nonionic surfactant and a balance of water, wherein the pharmaceutical composition has a pH of about 5.0 to about 6.2.

In some embodiments, the present application provides a pharmaceutical composition comprising about 0.2 mg/ml to about 20 mg/ml of the bispecific antibody, about 10 mM to about 50 mM of the buffer, about 100 mM to about 400 mM of the tonicity modifier selected from trehalose and mannitol, about 0.2 mM to about 5 mM of the metal ion chelator type antioxidant, about 0.05 mg/ml to about 1.0 mg/ml of the nonionic surfactant and a balance of water, wherein the pharmaceutical composition has a pH of about 5.0 to about 6.2.

In some embodiments, the present application provides a pharmaceutical composition comprising about 0.5 mg/ml to about 2.0 mg/ml of the bispecific antibody, about 10 mM to about 50 mM of the buffer, about 100 mM to about 400 mM of the tonicity modifier selected from trehalose and mannitol, about 0.2 mM to about 5 mM of the metal ion chelator type antioxidant, about 0.05 mg/ml to about 1.0 mg/ml of the nonionic surfactant and a balance of water, wherein the pharmaceutical composition has a pH of about 5.2 to about 6.2.

In some embodiments, the content of the bispecific antibody in the pharmaceutical composition is about 0.2 mg/ml to about 20 mg/ml, about 0.5 mg/ml to about 2.0 mg/ml, about 0.5 mg/ml to about 1.0 mg/ml, or about 1.0 mg/ml to about 2.0 mg/ml, or about 0.5 mg/ml, about 1.0 mg/ml, about 1.5 mg/ml or about 2.0 mg/ml. Alternatively, a person skilled in the art can select an appropriate content of the bispecific antibody according to practical needs.

In some embodiments, the buffer can be those commonly used in the art for preparing liquid formulations, examples of which include but are not limited to a buffer pair of phosphoric acid/phosphate, a buffer pair of histidine/an inorganic salt of histidine, a buffer pair of acetic acid/acetate, and a buffer pair of citric acid/citrate, and so on. In some embodiments, the buffer is a buffer pair of histidine/an inorganic salt of histidine, or a buffer pair of acetic acid/acetate, such as a buffer pair of histidine/histidine hydrochloride or a buffer pair of acetic acid/sodium acetate. In some embodiments, the buffer is the buffer pair of histidine/histidine hydrochloride.

In some embodiments, the concentration of the buffer in the pharmaceutical composition is about 10 mM to about 50 mM, about 15 mM to about 30 mM, about 15 mM to about 25 mM, about 15 mM to about 20 mM, or about 20 mM to about 25 mM, or about 20 mM. In some embodiments, the buffer is the buffer pair of histidine/histidine hydrochloride or the buffer pair of acetic acid/sodium acetate at a concentration of about 10 mM to about 50 mM, about 15 mM to about 30 mM, about 15 mM to about 25 mM, about 15 mM to about 20 mM, about 20 mM to about 25 mM, or about 20 mM. Alternatively, a person skilled in the art can select an appropriate concentration of the buffer pair of histidine/histidine hydrochloride or the buffer pair of acetic acid/sodium acetate according to practical needs. When the buffer pair of histidine/histidine hydrochloride and the buffer pair of acetic acid/sodium acetate are selected, the bispecific antibody in the pharmaceutical composition aggregates slowly, and the buffer can not only maintain the pH of the pharmaceutical composition, but also be quickly neutralized to physiological pH when entering the human body for clinical use.

In some embodiments, the concentration of the tonicity modifier in the pharmaceutical composition is about 100 mM to about 400 mM, about 200 mM to about 300 mM, about 200 mM to about 250 mM, about 230 mM to about 260 mM, or about 250 mM to about 300 mM, for example, about 200, 210, 220, 230, 240, 250, 260, 270, 280, 290 or 300 mM. The tonicity modifier is selected from trehalose and mannitol, preferably trehalose. Trehalose and mannitol have a solvent exclusion effect in the pharmaceutical formulation comprising the bispecific antibody of the present application, which will improve the stability of the bispecific antibody in an aqueous solution and inhibit the aggregation of the bispecific antibody.

In some embodiments, examples of the metal ion chelator type antioxidant in the pharmaceutical composition include but are not limited to, ethylenediamine tetraacetate salt, ethylene glycol tetraacetic acid (EGTA), thiamine tetrahydrofurfuryl disulfide (TTFD), 2,3-dimercaptosuccinic acid (DMSA), diethylene triaminepentaacetic acid or dimercaptopropanol. In some embodiments, ethylenediamine tetraacetate salt can be an alkali metal salt or an alkaline earth metal salt of ethylenediamine tetraacetic acid, such as disodium ethylenediamine tetraacetate or calcium sodium ethylenediamine tetraacetate. In some embodiments, the concentration of the metal ion chelator type antioxidant (e.g., ethylenediamine tetraacetate salt) is about 0.2 mM to about 5 mM, about 0.5 mM to about 5 mM, about 0.5 mM to about 2.0 mM, 0.5 mM to about 1.5 mM, about 0.5 mM to about 1.0 mM, or about 1.0 mM to about 1.5 mM or about 1.0 mM. In some embodiments, the concentration of the metal ion chelator type antioxidant (e.g., ethylenediamine tetraacetate salt) is about 0.5, about 1.0, about 1.5, about 2.0, about 2.5, about 3.0, about 3.5, about 4.0, about 4.5 or 5.0 mM. Alternatively, a person skilled in the art can select an appropriate concentration of the metal ion chelator type antioxidant (e.g., ethylenediamine tetraacetate salt) according to practical needs.

In some embodiments, the nonionic surfactant in the pharmaceutical composition can be a polysorbate-type surfactant, such as polysorbate 20 or polysorbate 80. In some embodiments, the content of the nonionic surfactant (e.g., a polysorbate-type surfactant, such as polysorbate 20 or polysorbate 80) is about 0.05 mg/ml to about 1.0 mg/ml, about 0.1 mg/ml to about 1.0 mg/ml, about 0.2 mg/ml to about 0.5 mg/ml, about 0.3 mg/ml to about 1.0 mg/ml, or about 0.5 mg/ml to about 1.0 mg/ml, for example, about 0.3 mg/ml, 0.34 mg/ml, about 0.4 mg/ml, 0.5 mg/ml, 0.6 mg/ml, 0.7 mg/ml, 0.8 mg/ml, 0.9 mg/ml or 1.0 mg/ml. The bispecific antibody as a protein may be denatured at an interface (air/liquid interface, liquid/glass interface, etc.) due to hydrophobic interaction, and thereby protein aggregation or even precipitation may occur. The use of the nonionic surfactant (for example, polysorbate-type surfactants, such as polysorbate 20 or polysorbate 80) can prevent protein aggregation and precipitation during shaking, stirring, or freezing and thawing.

In some embodiments, the pharmaceutical composition has a pH of about 5.0 to about 6.2, about 5.0 to about 6.0, about 5.2 to about 6.2, about 5.5 to about 6.0, or about 5.5 to about 5.8, such as about 5.0, 5.1, 5.2, 5.3, 5.4, 5.5, 5.6, 5.7, 5.8, 5.9 or 6.0.

In some embodiments, the water in the pharmaceutical composition may be sterile, pyrogen-free water, such as water for injection.

In some embodiments, the present application provides the pharmaceutical composition comprising about 0.5 mg/ml to about 2.0 mg/ml of the bispecific antibody, about 10 mM to about 50 mM of the buffer pair of histidine/histidine hydrochloride or the buffer pair of acetic acid/sodium acetate, about 100 mM to about 400 mM of the tonicity modifier selected from trehalose and mannitol, about 0.2 mM to about 5 mM of ethylenediamine tetraacetate salt, about 0.05 mg/ml to about 1.0 mg/ml of polysorbate 80 and a balance of water, wherein the pharmaceutical composition has the pH of about 5.5 to about 6.0.

In some embodiments, the present application provides the pharmaceutical composition comprising about 0.5 mg/ml to about 2.0 mg/ml of the bispecific antibody, about 15 mM to about 25 mM of the buffer pair of histidine/histidine hydrochloride or the buffer pair of acetic acid/sodium acetate, about 200 mM to about 300 mM of the tonicity modifier selected from trehalose and mannitol, about 0.5 mM to about 5 mM of ethylenediamine tetraacetate salt, about 0.3 mg/ml to about 1.0 mg/ml of polysorbate 80 and a balance of water, wherein the pharmaceutical composition has the pH of about 5.5 to about 6.0.

In some embodiments, the present application provides the pharmaceutical composition comprising about 0.5 mg/ml to about 2.0 mg/ml of the bispecific antibody, about 10 mM to about 50 mM of the buffer pair of histidine/histidine hydrochloride, about 100 mM to about 400 mM of trehalose, about 0.2 mM to about 5 mM of ethylenediamine tetraacetate salt, about 0.05 mg/ml to about 1.0 mg/ml of polysorbate 80 and a balance of water, wherein the pharmaceutical composition has the pH of about 5.5 to about 6.0.

In some embodiments, the present application provides the pharmaceutical composition comprising about 0.5 mg/ml to about 2.0 mg/ml of the bispecific antibody, about 15 mM to about 25 mM of the buffer pair of histidine/histidine hydrochloride, about 200 mM to about 300 mM of trehalose, about 0.5 mM to about 5 mM of ethylenediamine tetraacetate salt, about 0.3 mg/ml to about 1.0 mg/ml of polysorbate 80 and a balance of water, wherein the pharmaceutical composition has the pH of about 5.5 to about 6.0.

In some embodiments, the present application provides the pharmaceutical composition comprising about 0.5 mg/ml to about 2.0 mg/ml of the bispecific antibody, about 20 mM of the buffer pair of histidine/histidine hydrochloride, about 240 mM of trehalose, about 1.0 mM of ethylenediamine tetraacetate salt, about 0.5 mg/ml to about 1.0 mg/ml of polysorbate 80 and a balance of water, wherein the pharmaceutical composition has the pH of about 5.5 to about 5.8.

The pharmaceutical composition of the present application is a liquid formulation, which can be in the form of a solution, an emulsion or a suspension, preferably a solution. The pharmaceutical composition of the present application can be administered to a subject in need thereof via a parenteral administration route, such as an injection administration route. The injection administration route includes but is not limited to, subcutaneous injection (for example, subcutaneous infiltration injection into a wound), intramuscular injection, and intradermal injection.

After the pharmaceutical composition comprising the bispecific antibody of the present application was placed at 5° C.±3° C. for 6 months, SEC-HPLC monomer peak area %, CEX-HPLC acidic peak area %, CE-SDS (non-reducing method) and activity level of the composition were essentially unchanged. The pharmaceutical composition comprising the bispecific antibody of the present application can be stored for a long period of time at 5° C.±3° C. under the dark condition, and can maintain stable quality, thereby ensuring the safety, efficacy and uniformity of the pharmaceutical product.

It should be understood that the foregoing detailed description is intended only to enable those skilled in the art to have better understanding of the present application and is not intended to cause limitations in any way. Various modifications and variations can be made to the described embodiments by those skilled in the art.

The following Examples are for purposes of illustration only and are not intended to limit the scope of the present application. In the following examples, the S2E3-scFv-FcH+C34m-IgG1K antibody disclosed in CN201910706710.9 is used as an example of the bispecific antibody of the present application.

### EXAMPLES

### Example 1: Effect of Tonicity Modifiers on the Stability of Pharmaceutical Formulations Comprising the S2E3-SCFV-FCH+C34M-IGG1K Antibody

An appropriate amount of the S2E3-SCFV-FCH+C34M-IGG1K antibody sample was weighted and displaced into pure water by ultrafiltration, and then diluted to 2 mg/ml with purified water to obtain an aqueous solution of the S2E3-SCFV-FCH+C34M-IGG1K antibody. 2×formulation buffer stock solution (comprising 2× concentration of buffer and tonicity modifier, pH of about 6.0) of each formula as shown in Table 1 was prepared. Then, 7 ml of the aqueous solution of the S2E3-SCFV-FCH+C34M-IGG1K antibody, 7 ml of the 2×formulation buffer stock solution and 28 μL of 10% polysorbate 80 solution were taken and mixed uniformly to afford the formulation samples comprising the tonicity modifier selected from trehalose, mannitol, sucrose, arginine hydrochloride, glycine or proline, and the formulation sample without tonicity modifier, respectively.

TABLE 1
Formula compositions of liquid formulations

| | S2E3-SCFV-FCH + | | | | |
|---|---|---|---|---|---|
| | C34M-IGG1K antibody | | | Polysorbate | |
| Formulation | concentration | | Tonicity | 80 (0.2 | |
| No. | 1.0 mg/ml | Buffer | modifier | mg/ml) | pH |
| H60T | √ | 20 mM | 90 mg/ml | √ | 6.0 |
| | | histidine salt | trehalose | | |
| H60 | √ | 20 mM | — | √ | 6.0 |
| | | histidine salt | | | |
| H60M | √ | 20 mM | 45 mg/ml | √ | 6.0 |
| | | histidine salt | mannitol | | |
| H60S | √ | 20 mM | 80 mg/ml | √ | 6.0 |
| | | histidine salt | sucrose | | |
| H60R | √ | 20 mM | 32 mg/ml | √ | 6.0 |
| | | histidine salt | arginine | | |
| | | | hydrochloride | | |
| H60G | √ | 20 mM | 20 mg/ml | √ | 6.0 |
| | | histidine salt | glycine | | |
| H60P | √ | 20 mM | 30 mg/ml | √ | 6.0 |
| | | histidine salt | proline | | |

“√” indicates that the corresponding substance was comprised in the formulation; “—” indicates that the corresponding substance was not comprised in the formulation; and 20 mM histidine salt was a buffer system consisting of histidine and histidine hydrochloride, which contained 20 mM histidine/histidine ions.

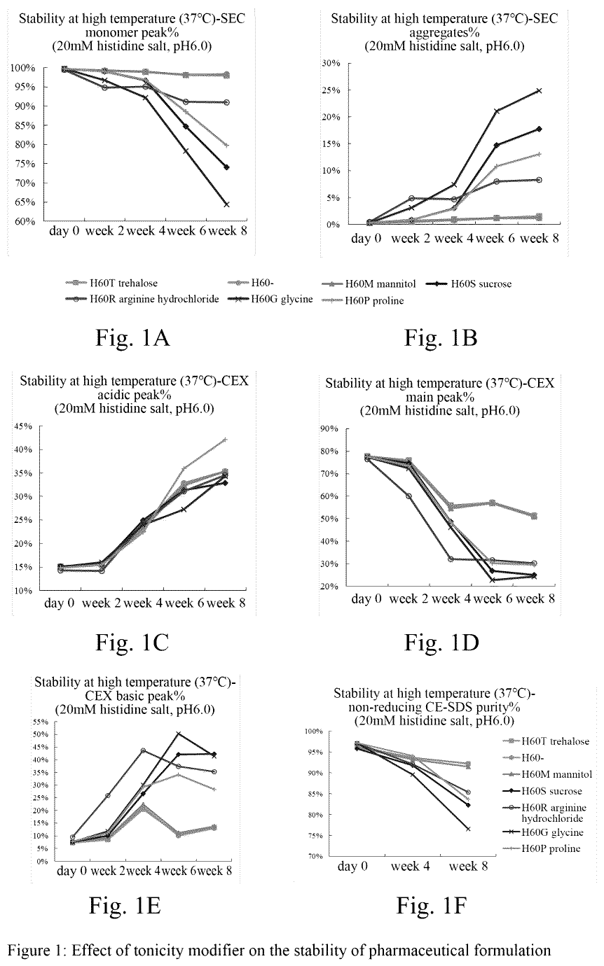

Samples of each formulation were stored under high-temperature (37±2° C.) condition for stability testing to accelerate the degradation of the S2E3-SCFV-FCH+C34M-IGG1K antibody. The samples were taken at day 0, week 2, week 4, week 6, and week 8, and the purity of the formulation samples at each time point was determined by SEC-HPLC, CEX-HPLC, non-reducing CE-SDS, and reducing CE-SDS methods as described below. The Tm value of each formulation sample was determined by the DSF method to determine conformational stability properties thereof. The detection results were shown in FIGS. 1A-1F.

SEC-HPLC (molecule exclusion chromatography) purity determination was conducted by high-performance liquid chromatography using TSKgel G3000SWXL chromatographic column and 50 mM PB/300 mM NaCl, pH 7.0 solution as the mobile phase. The samples were diluted, injected, and eluted with an isocratic elution at a flow rate of 1.0 ml/min for 15 minutes, and detected at a UV wavelength of 280 nm. The peak area percentages of monomers and polymers were obtained through peak area normalization.

CEX-HPLC (cation exchange chromatography) purity determination was conducted by high-performance liquid chromatography using MabPac™SCX-10 BioLC™ Analytical chromatographic column, 20 mM MES/pH5.6 solution as the mobile phase A and 20 mM MES/0.5M NaCl/pH5.6 solution as the mobile phase B. The samples were diluted, injected, and eluted with a gradient at a flow rate of 0.5 ml/min for 50 minutes, and detected at a UV wavelength of 280 nm. The peak area percentages of acidic peak, main peak and basic peak were obtained through peak area normalization.

The non-reducing CE-SDS (sodium dodecyl sulfate capillary electrophoresis) and reducing CE-SDS methods were carried out by a capillary electrophoresis instrument using an uncoated capillary column with an effective length of 20 cm and SDS-MW Gel separation gel at a column temperature of 25° C., a separation voltage of 15 kV, and a detection wavelength of 214 nm. The purity of the samples was determined under non-reducing and reducing conditions, respectively.

DSF (Differential Scanning Fluorescence) was determined using a Real-Time PCR instrument. The samples were diluted and mixed with a fluorescent dye (SYPRO®Orange Protein gel stain, Life), and then transferred to PCR tubes. The heating program was set up (from 25° C. to 95° C., 1° C./min). Fluorescence was collected in real time and data were recorded. The fluorescence-temperature curve was derived, and the highest peak of the curve after derivation was the Tm value.

TABLE 2
Effect of the tonicity modifiers on Tm values of pharmaceutical
formulations comprising the S2E3-SCFV-FCH+C34M-IGGIK antibody

| Formulation No. | H60T | H60 | H60M | H60S | H60R | H60G | H60P |
|---|---|---|---|---|---|---|---|
| Tₘ value (° C.) | 61.1 | 59.9 | 60.6 | 60.9 | 58.8 | 60.6 | 60.2 |

As can be seen from Table 2 and FIGS. 1A-1F, different tonicity modifiers had little effects on the Tm values of the pharmaceutical formulations comprising the S2E3-SCFV-FCH+C34M-IGG1K antibody. The changes of the Tm values were less than 2° C., and the Tm values were all at about 60° C. The Tm value of the formulation comprising arginine hydrochloride as the tonicity modifier was slightly lower. Under high-temperature stress conditions, sucrose, arginine hydrochloride, glycine, and proline increased the levels of SEC-HPLC aggregates and CEX-HPLC basic peaks of the liquid formulations comprising the S2E3-SCFV-FCH+C34M-IGG1K antibody relatively rapidly, and decreased the level of the non-reducing CE-SDS purity relatively quickly, and therefore, these tonicity modifiers were not suitable for use in the preparation of liquid formulations comprising the S2E3-SCFV-FCH+C34M-IGG1K antibody. Formulation samples comprising trehalose or mannitol as the tonicity modifiers were relatively stable under high-temperature stress conditions.

### Example 2: Effect of Antioxidants on the Stability of Pharmaceutical Formulations Comprising the S2E3-SCFV-FCH+C34M-IGG1K Antibody

Liquid formulations H60T, H60TE and H60TM were prepared using a preparation method similar to that described in Example 1 according to the formula compositions as shown in Table 3, expect for adding 1 mM disodium ethylenediamine tetraacetate or 1 mM methionine as antioxidants.

TABLE 3
Formula compositions of liquid formulations

| | S2E3-SCFV-FCH + C34M- | | | | Polysorbate | |
|---|---|---|---|---|---|---|
| Formulation | IGG1K antibody concentration | | Tonicity | | 80 | |
| No. | 1.0 mg/ml | Buffer | modifier | Antioxidant | (0.2 mg/ml) | pH |
| H60T | ✓ | 20 mM | 90 mg/ml | — | ✓ | 6.0 |
| | | histidine salt | trehalose | | | |
| H60TE | ✓ | 20 mM | 90 mg/ml | 1 mM disodium | ✓ | 6.0 |
| | | histidine salt | trehalose | ethylenediamine | | |
| | | | | tetraacetate | | |
| H60TM | ✓ | 20 mM | 90 mg/ml | 1 mM methionine | ✓ | 6.0 |
| | | histidine salt | trehalose | | | |

“✓” indicates that the corresponding substance was comprised in the formulation;
“—” indicates that the corresponding substance was not comprised in the formulation; and 20 mM histidine salt was a buffer system consisting of histidine and histidine hydrochloride, which contained 20 mM histidine/histidine ions.

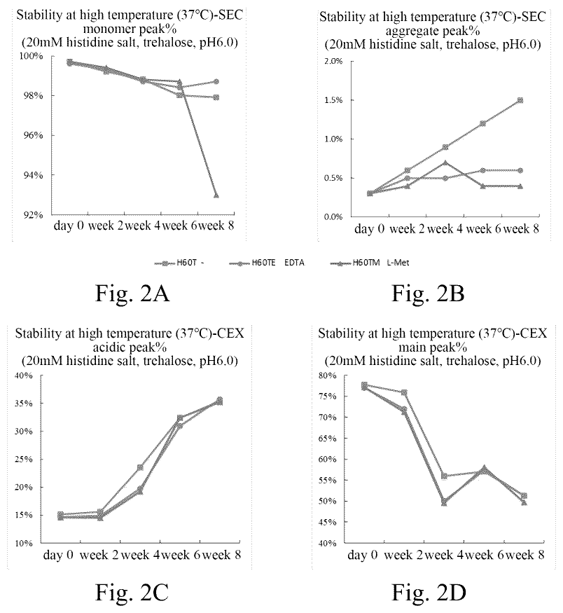

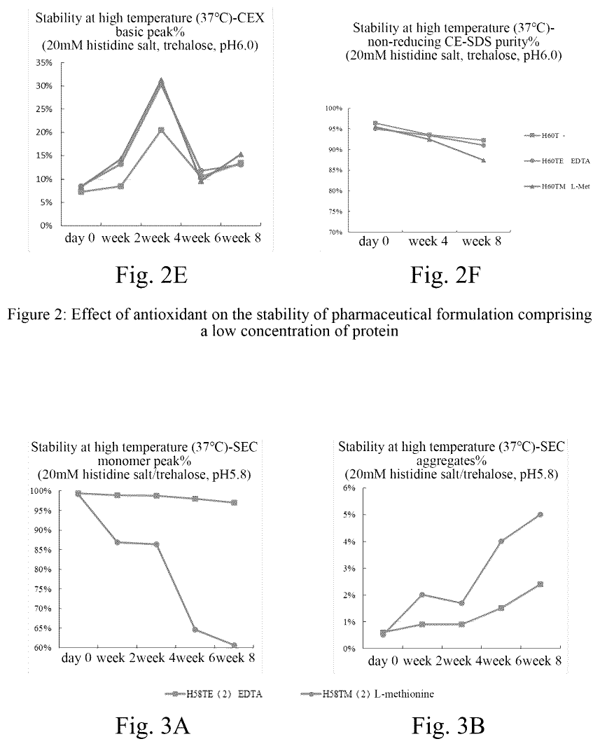

The purity of each formulation sample was determined by using stress conditions, sampling time points and test items similar to those described in Example 1. The test results were shown in Table 4 and FIGS. 2A-2F.

TABLE 4
Effect of antioxidants on the Tm values of pharmaceutical formulations
comprising the S2E3-SCFV-FCH + C34M-IGG1K antibody

| Formulation No. | H60T | H60TE | H60TM |
|---|---|---|---|
| Tm value (° C.) | 61.1 | 61.1 | 61.2 |

As can be seen from Table 4 and FIGS. 2A-2F, the addition of antioxidants did not affect the Tm values of the pharmaceutical formulations comprising the S2E3-SCFV-FCH+C34M-IGG1K antibody. Both disodium ethylenediamine tetraacetate and methionine reduced the rate of increase in SEC aggregates, and did not change the trend of decrease in the purity of CEX. However, methionine accelerated the rate of decrease in the purity of non-reducing CE-SDS.

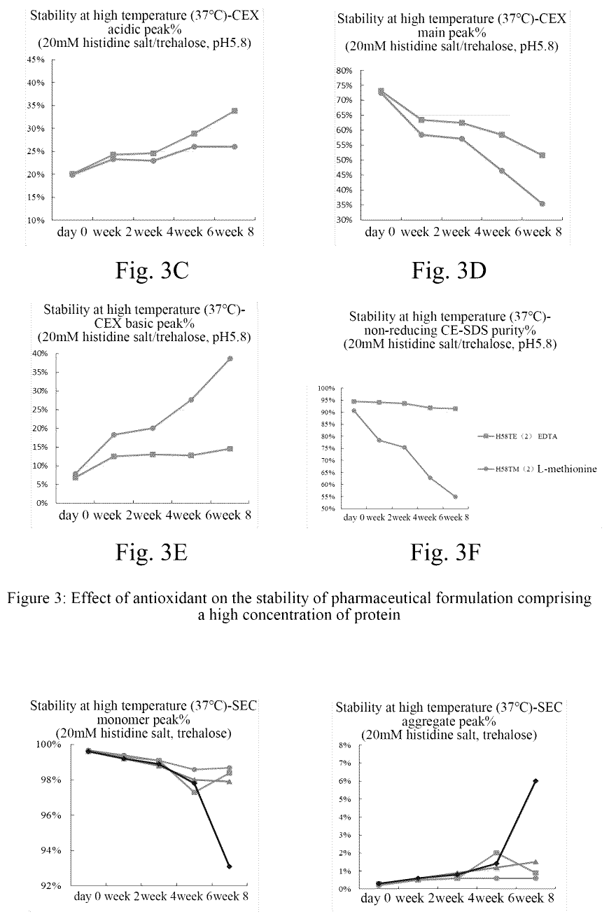

The effect of antioxidants on the stability of the pharmaceutical formulations comprising the S2E3-SCFV-FCH+C34M-IGG1K antibody was further investigated by repeating the above experimental steps and using the following formula compositions. The test results were shown in FIGS. 3A-3F.

TABLE 5
Formula compositions of liquid formulations

| | S2E3-SCFV-FCH + C34M- | | | | Polysorbate | |
|---|---|---|---|---|---|---|
| Formulation | IGG1K antibody concentration | | Tonicity | | 80 | |
| No. | 2.0 mg/ml | Buffer | modifier | Antioxidant | (0.5 mg/ml) | pH |
| H58TE(2) | ✓ | 20 mM | 90 mg/ml | 1 mM disodium | ✓ | 5.8 |
| | | histidine salt | trehalose | Ethylenediamine | | |
| | | | | tetraacetate | | |
| H58TM(2) | ✓ | 20 mM | 90 mg/ml | 1 mM methionine | ✓ | 5.8 |
| | | histidine salt | trehalose | | | |

“✓” indicates that the corresponding substance was comprised in the formulation; and 20 mM histidine salt was a buffer system consisting of histidine and histidine hydrochloride, which contained 20 mM histidine/histidine ions.

As can be seen from FIGS. 3A-3F, for the pharmaceutical formulation comprising a higher concentration of the S2E3-SCFV-FCH+C34M-IGG1K antibody, the purities of SEC-HPLC, CEX-HPLC and non-reducing CE-SDS all decreased at an accelerated rate at a high temperature (37±2° C.) when L-methionine was used as an antioxidant.

It can be concluded that disodium ethylenediamine tetraacetate was more suitable as an antioxidant in the pharmaceutical formulations comprising the S2E3-SCFV-FCH+C34M-IGG1K antibody, compared with L-methionine.

### Example 3: Effect of pH Values of Pharmaceutical Formulations on the Stability of Pharmaceutical Formulations Comprising the S2E3-SCFV-FCH+C34M-IGG1K Antibody

Liquid formulations H55T, H58T, H60T and H62T were prepared using a preparation method similar to that described in Example 1 according to the formula compositions as shown in Table 6, except that the final pH values of the formulations were adjusted to 5.5-6.2.

TABLE 6
Formula compositions of liquid formulations

| | S2E3-SCFV-FCH + | | | | |
|---|---|---|---|---|---|
| | C34M-IGG1K antibody | | | Polysorbate | |
| Formulation | concentration | | Tonicity | 80 (0.2 | |
| No. | 1.0 mg/ml | Buffer | modifier | mg/ml) | pH |
| H60T | √ | 20 mM | 90 mg/ml | √ | 6.0 |
| | | histidine salt | trehalose | | |
| H55T | √ | 20 mM | 90 mg/ml | √ | 5.5 |
| | | histidine salt | trehalose | | |
| H58T | √ | 20 mM | 90 mg/ml | √ | 5.8 |
| | | histidine salt | trehalose | | |
| H62T | √ | 20 mM | 90 mg/ml | √ | 6.2 |
| | | histidine salt | trehalose | | |

“√” indicates that the corresponding substance was comprised in the formulation; and 20 mM histidine salt was a buffer system consisting of histidine and histidine hydrochloride, which contained 20 mM histidine/histidine ions.

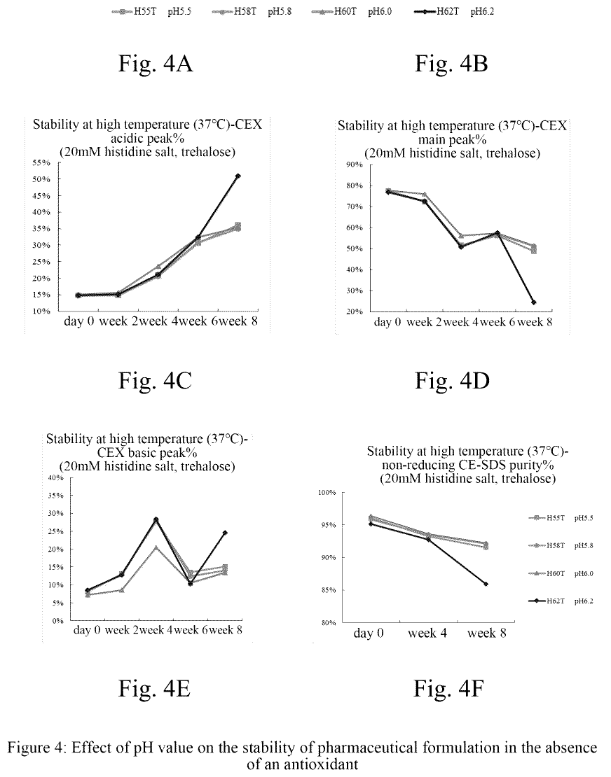

The purity of each formulation sample was determined by using stress conditions, sampling time points and test items similar to those described in Example 1. The test results were shown in Table 7 and FIGS. 4A-4F.

TABLE 7
Effect of pH values of pharmaceutical formulations
on the Tm values of pharmaceutical formulations
comprising the S2E3-SCFV-FCH + C34M-IGG1K antibody

| Formulation No. | H55T | H58T | H60T | H62T |
|---|---|---|---|---|
| Tm value (° C.) | 60.5 | 60.9 | 61.1 | 60.9 |

As can be seen from Table 7 and FIGS. 4A-4F, the Tm values of pharmaceutical formulations comprising the S2E3-SCFV-FCH+C34M-IGG1K antibody were not affected substantially by changes in the pH value of the formulations. In the range of pH 5.5-6.2, under high-temperature stress conditions, except for the formulation at pH 6.2, which exhibited a faster decrease in purity, the formulation at pH 5.8 exhibited the slowest increase in aggregates, while the formulations at pH 5.5, 5.8 and 6.0 exhibited almost no difference in the trend of decrease in the purity of CEX-HPLC and non-reducing CE-SDS.

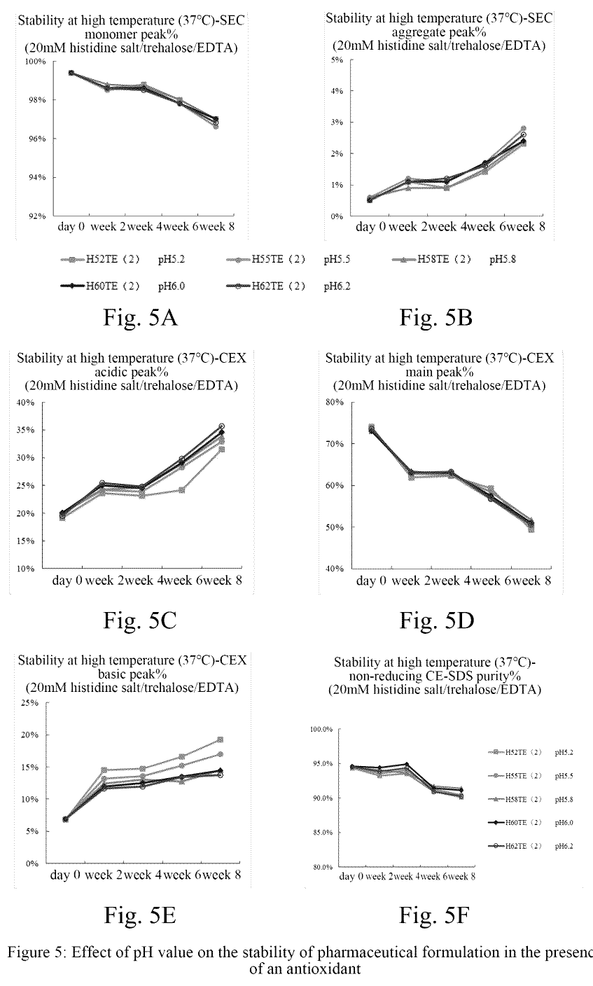

The effect of pH values of the pharmaceutical formulations on the stability of the pharmaceutical formulations comprising the S2E3-SCFV-FCH+C34M-IGG1K antibody in the presence of an antioxidant was further investigated by repeating the above experimental steps and using the following formula compositions. The test results were shown in FIGS. 5A-5F.

TABLE 8
Formula compositions of liquid formulations

| | S2E3-SCFV-FCH + C34M- | | | | Polysorbate | |
|---|---|---|---|---|---|---|
| Formulation | IGG1K antibody concentration | | Tonicity | | 80 | |
| No. | 2.0 mg/ml | Buffer | modifier | Antioxidant | (0.5 mg/ml) | PH |
| H52TE(2) | ✓ | 20 mM | 90 mg/ml | 1 mM disodium | ✓ | 5.2 |
| | | histidine salt | trehalose | ethylenediamine | | |
| | | | | tetraacetate | | |
| H55TE(2) | ✓ | 20 mM | 90 mg/ml | 1 mM disodium | ✓ | 5.5 |
| | | histidine salt | trehalose | ethylenediamine | | |
| | | | | tetraacetate | | |
| H58TE(2) | ✓ | 20 mM | 90 mg/ml | 1 mM disodium | ✓ | 5.8 |
| | | histidine salt | trehalose | ethylenediamine | | |
| | | | | tetraacetate | | |
| H60TE(2) | ✓ | 20 mM | 90 mg/ml | 1 mM disodium | ✓ | 6.0 |
| | | histidine salt | trehalose | ethylenediamine | | |
| | | | | tetraacetate | | |
| H62TE(2) | ✓ | 20 mM | 90 mg/ml | 1 mM disodium | ✓ | 6.2 |
| | | histidine salt | trehalose | ethylenediamine | | |
| | | | | tetraacetate | | |

“✓” indicates that the corresponding substance was comprised in the formulation; and 20 mM histidine salt was a buffer system consisting of histidine and histidine hydrochloride, which contained 20 mM histidine/histidine ions.

As can be seen from FIGS. 5A-5F, there was substantially no difference in the trend of decrease in the purity of SEC-HPLC and non-reducing CE-SDS in the range of pH 5.2-6.2. The rate of increase in the CEX basic peak was slightly faster for the formulation samples at pH 5.2 and pH 5.5. Therefore, the stability of the pharmaceutical formulations comprising the S2E3-SCFV-FCH+C34M-IGG1K antibody was not significantly affected in the range of pH 5.2-6.2.

### Example 4: Effect of Buffer Systems on the Stability of Pharmaceutical Formulations Comprising the S2E3-SCFV-FCH+C34M-IGG1K Antibody

Liquid formulations H58T, A50T, A52T, A55T and A58T were prepared using a preparation method similar to that described in Example 1 according to the formula compositions as shown in Table 9, except that an acetate buffer system was used instead of the histidine buffer system and the final pH values of the formulations were adjusted to 5.0-5.8.

TABLE 9
Formula compositions of liquid formulations

| | S2E3-SCFV-FCH + | | | | |
|---|---|---|---|---|---|
| | C34M-IGG1K antibody | | | Polysorbate | |
| Formulation | concentration | | Tonicity | 80 (0.2 | |
| No. | 1.0 mg/ml | Buffer | modifier | mg/ml) | pH |
| H58T | √ | 20 mM | 90 mg/ml | √ | 5.8 |
| | | histidine salt | trehalose | | |
| A50T | √ | 20 mM | 90 mg/ml | √ | 5.0 |
| | | acetate | trehalose | | |
| A52T | √ | 20 mM | 90 mg/ml | √ | 5.2 |
| | | acetate | trehalose | | |
| A55T | √ | 20 mM | 90 mg/ml | √ | 5.5 |
| | | acetate | trehalose | | |
| A58T | √ | 20 mM | 90 mg/ml | √ | 5.8 |
| | | acetate | trehalose | | |

“√” indicates that the corresponding substance was comprised in the formulation; 20 mM histidine salt was a buffer system consisting of histidine and histidine hydrochloride, which contained 20 mM histidine/histidine ions; and 20 mM acetate was a buffer system consisting of glacial acetic acid and sodium acetate, which contained 20 mM acetic acid/acetate ions.

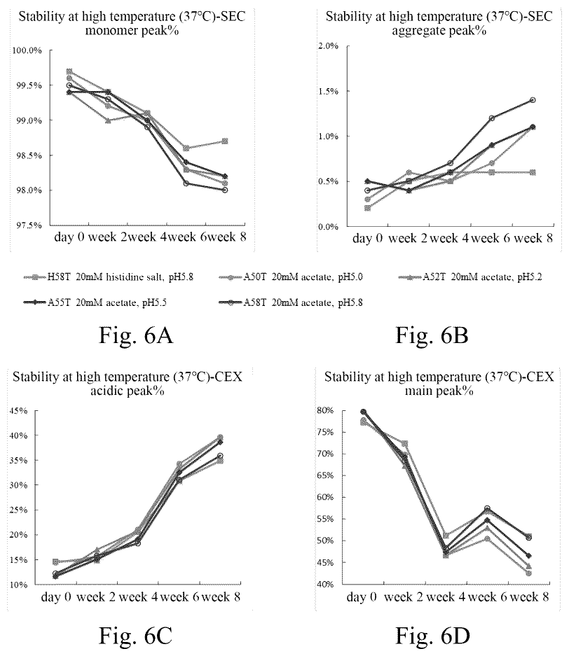

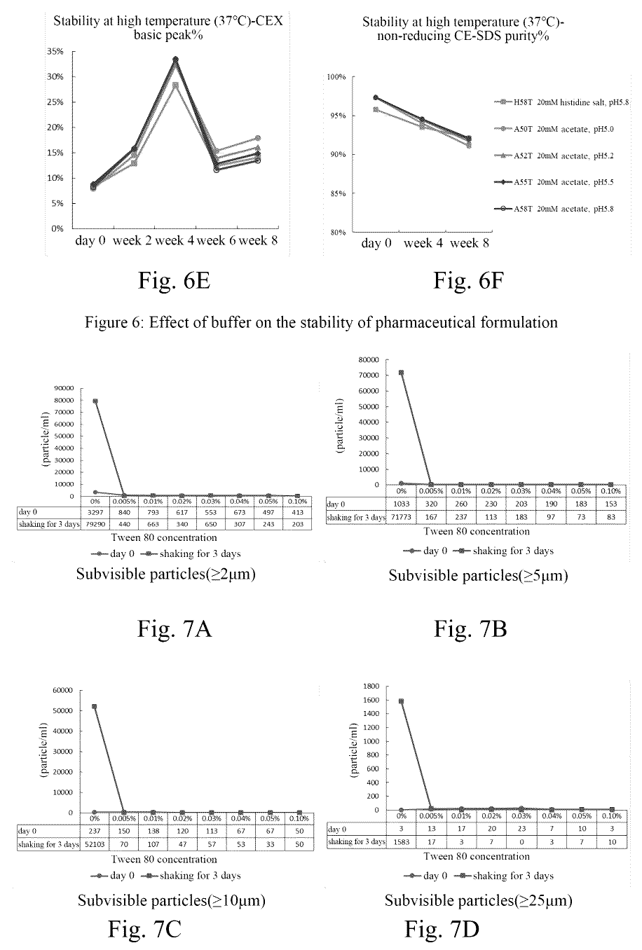

The stability of each formulation sample was determined by using stress conditions, sampling time points and test items similar to those described in Example 1. The test results were shown in Table 10 and FIGS. 6A-6F.

TABLE 10
Effect of buffer systems on the Tm values of pharmaceutical formulations
comprising the S2E3-SCFV-FCH + C34M-IGG1K antibody

| Formulation No. | H58T | A50T | A52T | A55T | A58T |
|---|---|---|---|---|---|
| Tm value (° C.) | 60.9 | 61.1 | 61.2 | 60.9 | 60.4 |

As can be seen from Table 10 and FIGS. 6A-6F, there was substantially no difference in the Tm values of the pharmaceutical formulations comprising the S2E3-SCFV-FCH+C34M-IGG1K antibody between the acetate buffer and histidine salt buffer. After the pharmaceutical formulations were placed at 37° C. for 8 weeks, the SEC aggregates of the S2E3-SCFV-FCH+C34M-IGG1K antibody increased slightly more rapidly in the acetate buffer in the range of pH 5.0-5.8 than in the histidine salt buffer at pH 5.8. However, there was substantially no difference in the trend of decrease in the purity of CEX-HPLC and non-reducing CE-SDS between the two buffer systems.

### Example 5: Effect of Polysorbate 80 on the Stability of Pharmaceutical Formulations Comprising the S2E3-SCFV-FCH+C34M-IGG1K Antibody

An appropriate amount of the S2E3-SCFV-FCH+C34M-IGG1K antibody sample was weighted, and displaced into a buffer system (20 mM histidine/histidine hydrochloride, pH 5.8) by ultrafiltration, and then diluted to 2.5 mg/ml with the buffer system to obtain a solution of the S2E3-SCFV-FCH+C34M-IGG1K antibody. 5×formulation buffer stock solution (comprising 1× concentration of the buffer and 5× concentration of trehalose and disodium ethylenediamine tetraacetate, pH about 5.8) of each formula as shown in Table 11 was prepared. Then, 32 ml of the solution of the S2E3-SCFV-FCH+C34M-IGG1K antibody and 8 ml of the 5×formulation buffer stock solution were taken and mixed uniformly. 5 ml of the mixture was taken, and a certain volume of 10% polysorbate 80 was added to afford formulation samples comprising 0%, 0.005%, 0.01%, 0.02%, 0.03%, 0.04%, 0.05%, and 0.10% polysorbate 80, respectively.

TABLE 11
Formula compositions

| S2E3-SCFV-FCH + | | | | Polysorbate 80 | |
|---|---|---|---|---|---|
| C34M-IGG1K antibody | | Tonicity | | concentration | |
| concentration | Buffer | modifier | Antioxidant | (w/v) | pH |
| 2.0 mg/ml | 20 mM | 90 mg/ml | 1 mM | 0% | 5.8 |
| | histidine/ | trehalose | disodium | 0.005% | |
| | histidine | | ethylenediamine | 0.01% | |
| | hydrochloride | | tetraacetate | 0.02% | |
| | | | | 0.03% | |
| | | | | 0.04% | |
| | | | | 0.05% | |
| | | | | 0.10% | |

“%” in Table 11 means weight/volume percentage or % w/v. For example, 0.05% polysorbate 80 means that the formulation comprises 0.5 mg/mL of polysorbate 80. In addition, the formulation comprising 0.05% polysorbate 80 in Table 11 is numbered as H58TE (2) 0.05% Tween 80, and the formulation not comprising polysorbate 80 in Table 11 is numbered as H58TE-ps (2) without Tween 80.

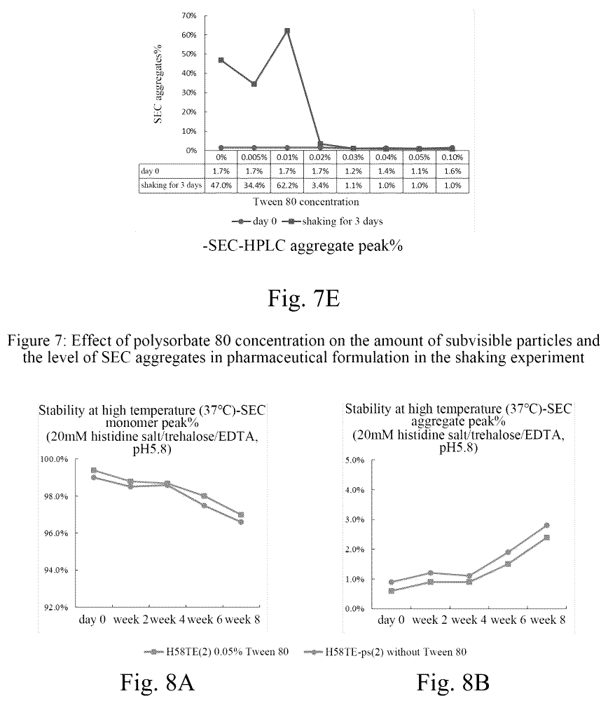

Each formulation sample in Table 11 was subjected to a shaking experiment. After shaking for 3 days at a rate of 1500 times/min, the amount of subvisible particles (photoresist method) and the level of SEC-HPLC aggregates in each sample were detected. The results of the shaking experiment were shown in FIGS. 7A-7E. When the formulation sample did not comprise polysorbate 80, the amount of subvisible particles and the proportion of SEC aggregates in the sample increased significantly after shaking for 3 days, indicating that the formulations comprising the S2E3-SCFV-FCH+C34M-IGG1K antibody were susceptible to mechanical forces such as shaking, thereby resulting in protein aggregation. When polysorbate 80 at a concentration of 0.005% to 0.10% was added to a formulation comprising the S2E3-SCFV-FCH+C34M-IGG1K antibody, the amount of subvisible particles in the samples did not substantially increase after shaking for 3 days. The level of SEC-HPLC aggregates in the formulation samples comprising polysorbate 80 at a concentration of 0.005% to 0.02% was elevated after shaking. The proportion of SEC-HPLC aggregates in the formulation samples was substantially not elevated when the concentration of polysorbate 80 in the formulations was elevated to 0.03% and above.

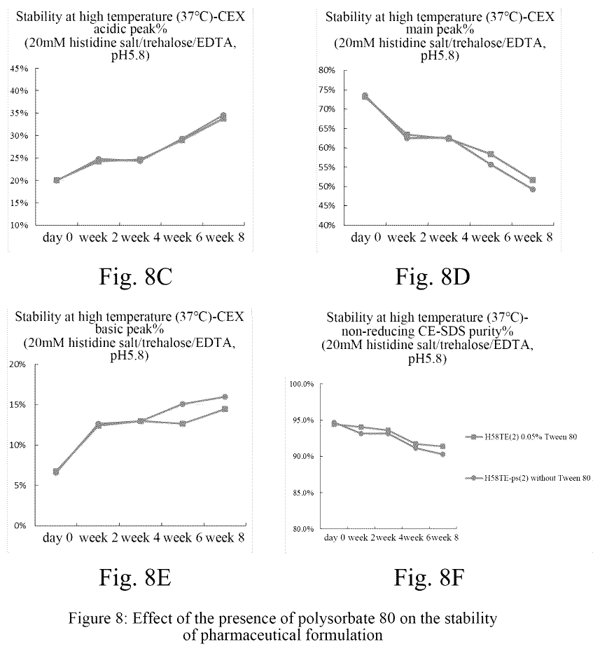

The stability of the formulation samples numbered as H58TE (2) 0.05% Tween 80 and H58TE-ps (2) without Tween 80 was determined using stress conditions, sampling time points and test items similar to those described in Example 1 to investigate the effect of degradation products that may be produced by polysorbate 80 on the stability of the formulations during stress. The results of said experiment were shown in FIGS. 8A-8F. There was substantially no difference in the trend of decrease in the purity of SEC-HPLC, CEX-HPLC and non-reducing CE-SDS between the formulation sample comprising 0.5 mg/ml polysorbate 80 and the formulation sample not comprising polysorbate 80 at a temperature of 37° C., indicating that the stability of pharmaceutical formulations comprising the S2E3-SCFV-FCH+C34M-IGG1K antibody was substantially unaffected by degradation products that may be produced by polysorbate 80 during stress.

### Example 6: Effect of Protein Concentrations on the Stability of Pharmaceutical Formulations Comprising the S2E3-SCFV-FCH+C34M-IGG1K Antibody

An appropriate amount of the S2E3-SCFV-FCH+C34M-IGG1K antibody stock solution (containing 20 mM histidine/histidine hydrochloride, 90 mg/ml trehalose, 1 mM disodium ethylenediamine tetraacetate, and 0.5 mg/ml polysorbate 80, pH 5.8) was taken, and respectively diluted to 20 mg/ml, 1 mg/ml, 0.5 mg/ml, and 0.2 mg/ml by using a buffer system, and mixed well to prepare formulation samples with different protein concentrations.

TABLE 12
Formula compositions

| | Protein | | Tonicity | | Polysorbate | |
|---|---|---|---|---|---|---|
| Formulation No. | concentration | Buffer | modifier | Antioxidant | 80 | pH |
| H58TE (0.2) | 0.2 mg/ml | 20 mM | 90 mg/ml | 1 mM disodium | 0.5 mg/ml | 5.8 |
| H58TE (0.5) | 0.5 mg/ml | histidine/histidine | trehalose | ethylenediamine | | |
| H58TE (1) | 1.0 mg/ml | hydrochloride | | tetraacetate | | |
| H58TE (20) | 20 mg/ml | | | | | |

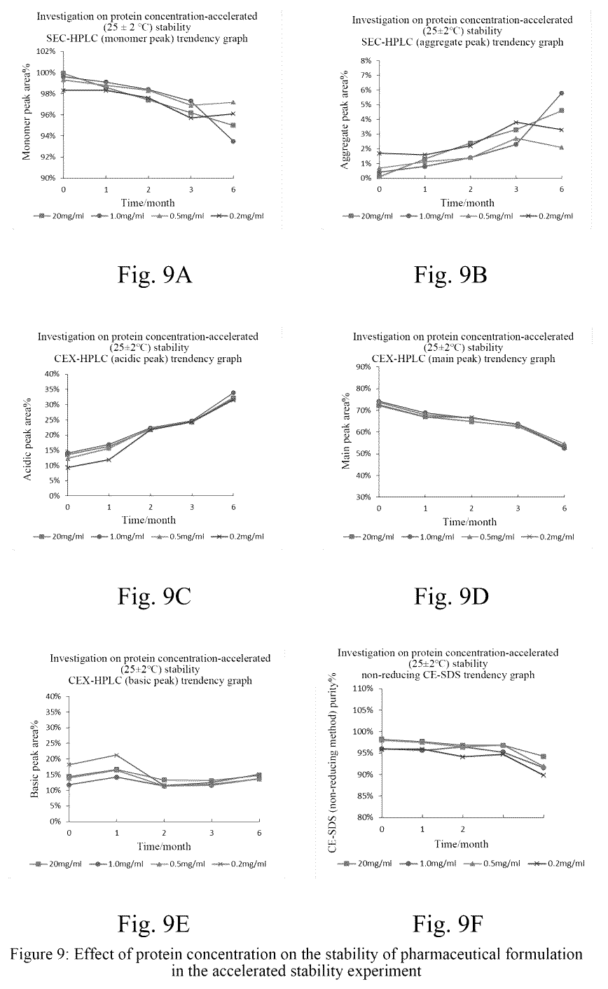

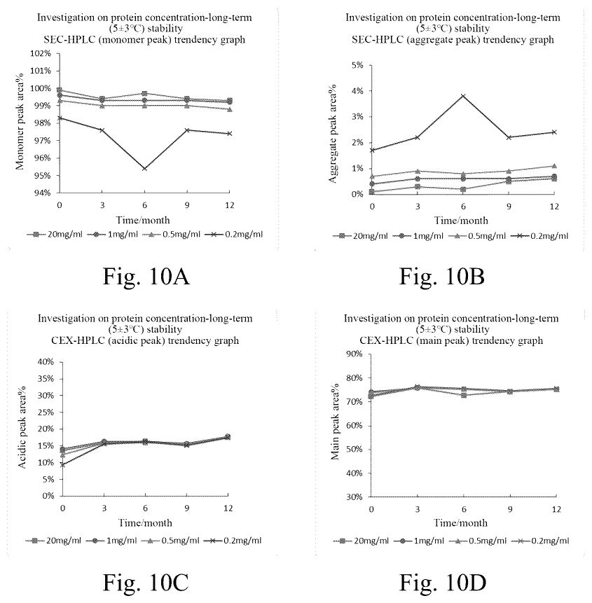

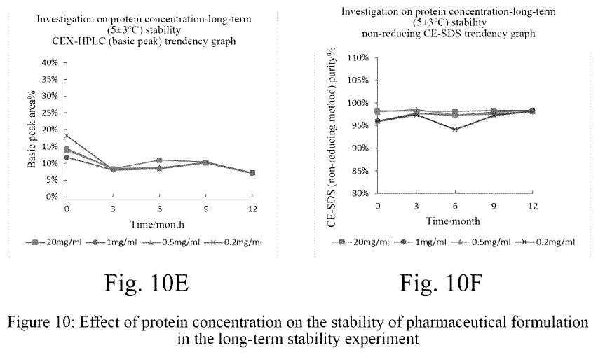

Each formulation sample was stored at 25±2° C. for accelerated stability testing and sampled at day 0, month 1, month 2 month, month 3 and month 6, and the levels of SEC-HPLC, CEX-HPLC and non-reducing CE-SDS in each sample were determined by using detection conditions similar to those described in Example 1. The test results were shown in Table 13 and FIGS. 9A-9F. In addition, each formulation sample was also stored at 5±3° C. for long-term stability testing, and sampled at day 0, month 3, month 6, month 9, and month 12, and the levels of SEC-HPLC, CEX-HPLC and non-reducing CE-SDS in each sample were determined by using detection conditions similar to those described in Example 1. The test results were shown in FIGS. 10A-10F.

As can be seen from FIGS. 9A-10F, under accelerated (25±2° C.) conditions, the formulation samples comprising the S2E3-SCFV-FCH+C34M-IGG1K antibody at concentrations of 0.2 mg/ml and 20 mg/ml exhibited a faster increase in the proportion of SEC aggregates during the first three months, and the trend of change in the purity of CEX-HPLC and non-reducing CE-SDS was consistent among the formulation samples with four different protein concentrations. At the temperature of 5±3° C., the formulation sample comprising the S2E3-SCFV-FCH+C34M-IGG1K antibody at a concentration of 0.2 mg/ml had a higher proportion of SEC aggregates, while the formulation samples comprising the S2E3-SCFV-FCH+C34M-IGG1K antibody at concentrations of 0.5 mg/ml, 1.0 mg/ml and 20 mg/ml had substantially the same trend of change in the proportion of SEC aggregates. The trends of change in the purity of CEX-HPLC and non-reducing CE-SDS were substantially consistent among the formulation samples with four different protein concentrations.

It can be seen that there was no difference in the change in the purity of CEX-HPLC and non-reducing CE-SDS when the formulation samples comprising the S2E3-SCFV-FCH+C34M-IGG1K antibody at concentrations of 0.2 mg/ml, 0.5 mg/ml, 1.0 mg/ml and 20 mg/ml were placed under accelerated (25±2° C.) and long-term (5±3° C.) conditions, while the formulation sample comprising the S2E3-SCFV-FCH+C34M-IGG1K antibody at a concentration of 0.2 mg/ml had a higher level of aggregates under long-term (5±3° C.) conditions.

1. A pharmaceutical composition, comprising about 0.2 mg/ml to about 20 mg/ml of a bispecific antibody against rabies virus G protein, a tonicity modifier selected from trehalose and mannitol, a metal ion chelator type antioxidant, a nonionic surfactant and a balance of water, wherein the pharmaceutical composition has a pH of about 5.0 to about 6.2, and wherein the bispecific antibody comprises an antigen-binding fragment that binds to epitope I of the rabies virus G protein and an antigen-binding fragment that binds to epitope III of the rabies virus G protein, and the bispecific antibody has the activity of neutralizing rabies virus.

2. The pharmaceutical composition according to claim 1, further comprising a buffer, such as about 10 mM to about 50 mM of the buffer, about 15 mM to about 30 mM of the buffer, about 15 mM to about 25 mM of the buffer, about 15 mM to about 20 mM of the buffer, about 20 mM to about 25 mM of the buffer, or about 20 mM of the buffer.

3. The pharmaceutical composition according to claim 2, wherein the pharmaceutical composition comprises about 0.5 mg/ml to about 2.0 mg/ml of the bispecific antibody against rabies virus G protein, about 10 mM to about 50 mM of the buffer, about 100 mM to about 400 mM of the tonicity modifier selected from trehalose and mannitol, about 0.2 mM to about 5 mM of the metal ion chelator type antioxidant (for example, ethylenediamine tetraacetate salt), about 0.05 mg/ml to about 1.0 mg/ml of the nonionic surfactant and a balance of water, wherein the pharmaceutical composition has a pH of about 5.2 to about 6.2.

4. The pharmaceutical composition according to claim 2, wherein the buffer is a buffer pair of phosphoric acid/phosphate, a buffer pair of histidine/an inorganic salt of histidine, a buffer pair of acetic acid/acetate, or a buffer pair of citric acid/citrate, preferably a buffer pair of histidine/an inorganic salt of histidine, or a buffer pair of acetic acid/acetate, more preferably a buffer pair of histidine/hydrochloride histidine or a buffer pair of acetic acid/sodium acetate, even more preferably a buffer pair of histidine/hydrochloride histidine.

5. (canceled)

6. The pharmaceutical composition according to claim 1, wherein a concentration of the tonicity modifier is about 200 mM to about 300 mM, about 200 mM to about 250 mM, about 230 mM to about 260 mM, or about 250 mM to about 300 mM.

7. The pharmaceutical composition according to claim 1, wherein a concentration of the metal ion chelator type antioxidant (for example, ethylenediamine tetraacetate salt) is about 0.5 mM to about 2.0 mM, about 0.5 mM to about 1.5 mM, about 0.5 mM to about 1.0 mM, or about 1.0 mM to about 1.5 mM, or about 1.0 mM.

8. The pharmaceutical composition according to claim 1, wherein the nonionic surfactant is a polysorbate type surfactant, such as polysorbate 20 or polysorbate 80, and an amount of the nonionic surfactant is about 0.1 mg/ml to about 1.0 mg/ml, about 0.2 mg/ml to about 0.5 mg/ml, about 0.3 mg/ml to about 1.0 mg/ml, or about 0.5 mg/ml to about 1.0 mg/ml, for example, about 0.3 mg/ml, about 0.34 mg/ml, about 0.4 mg/ml, about 0.5 mg/ml, about 0.6 mg/ml, about 0.7 mg/ml, about 0.8 mg/ml, about 0.9 mg/ml or about 1.0 mg/ml.

9. The pharmaceutical composition according to claim 1, wherein the pharmaceutical composition has the pH of about 5.0 to about 6.0, about 5.2 to about 6.2, about 5.5 to about 6.0, about 5.5 to about 5.8, or about 5.8

10. (canceled)

11. A method for preventing or treating rabies in a subject in need thereof, comprising administering the pharmaceutical composition of claim 1 to the subject.

12.-13. (canceled)

14. The pharmaceutical composition according to claim 3, wherein the buffer is a buffer pair of phosphoric acid/phosphate, a buffer pair of histidine/an inorganic salt of histidine, a buffer pair of acetic acid/acetate, or a buffer pair of citric acid/citrate, preferably a buffer pair of histidine/an inorganic salt of histidine, or a buffer pair of acetic acid/acetate, more preferably a buffer pair of histidine/hydrochloride histidine or a buffer pair of acetic acid/sodium acetate, even more preferably a buffer pair of histidine/hydrochloride histidine.

15. The pharmaceutical composition according to claim 2, wherein a concentration of the tonicity modifier is about 200 mM to about 300 mM, about 200 mM to about 250 mM, about 230 mM to about 260 mM, or about 250 mM to about 300 mM.

16. The pharmaceutical composition according to claim 3, wherein a concentration of the tonicity modifier is about 200 mM to about 300 mM, about 200 mM to about 250 mM, about 230 mM to about 260 mM, or about 250 mM to about 300 mM.

17. The pharmaceutical composition according to claim 4, wherein a concentration of the tonicity modifier is about 200 mM to about 300 mM, about 200 mM to about 250 mM, about 230 mM to about 260 mM, or about 250 mM to about 300 mM.

18. The pharmaceutical composition according to claim 2, wherein a concentration of the metal ion chelator type antioxidant (for example, ethylenediamine tetraacetate salt) is about 0.5 mM to about 2.0 mM, about 0.5 mM to about 1.5 mM, about 0.5 mM to about 1.0 mM, or about 1.0 mM to about 1.5 mM, or about 1.0 mM.

19. The pharmaceutical composition according to claim 3, wherein a concentration of the metal ion chelator type antioxidant (for example, ethylenediamine tetraacetate salt) is about 0.5 mM to about 2.0 mM, about 0.5 mM to about 1.5 mM, about 0.5 mM to about 1.0 mM, or about 1.0 mM to about 1.5 mM, or about 1.0 mM.

20. The pharmaceutical composition according to claim 4, wherein a concentration of the metal ion chelator type antioxidant (for example, ethylenediamine tetraacetate salt) is about 0.5 mM to about 2.0 mM, about 0.5 mM to about 1.5 mM, about 0.5 mM to about 1.0 mM, or about 1.0 mM to about 1.5 mM, or about 1.0 mM.

21. The pharmaceutical composition according to claim 2, wherein the nonionic surfactant is a polysorbate type surfactant, such as polysorbate 20 or polysorbate 80, and an amount of the nonionic surfactant is about 0.1 mg/ml to about 1.0 mg/ml, about 0.2 mg/ml to about 0.5 mg/ml, about 0.3 mg/ml to about 1.0 mg/ml, or about 0.5 mg/ml to about 1.0 mg/ml, for example, about 0.3 mg/ml, about 0.34 mg/ml, about 0.4 mg/ml, about 0.5 mg/ml, about 0.6 mg/ml, about 0.7 mg/ml, about 0.8 mg/ml, about 0.9 mg/ml or about 1.0 mg/ml.

22. The pharmaceutical composition according to claim 3, wherein the nonionic surfactant is a polysorbate type surfactant, such as polysorbate 20 or polysorbate 80, and an amount of the nonionic surfactant is about 0.1 mg/ml to about 1.0 mg/ml, about 0.2 mg/ml to about 0.5 mg/ml, about 0.3 mg/ml to about 1.0 mg/ml, or about 0.5 mg/ml to about 1.0 mg/ml, for example, about 0.3 mg/ml, about 0.34 mg/ml, about 0.4 mg/ml, about 0.5 mg/ml, about 0.6 mg/ml, about 0.7 mg/ml, about 0.8 mg/ml, about 0.9 mg/ml or about 1.0 mg/ml.

23. The pharmaceutical composition according to claim 2, wherein the pharmaceutical composition has the pH of about 5.0 to about 6.0, about 5.2 to about 6.2, about 5.5 to about 6.0, about 5.5 to about 5.8, or about 5.8.

24. The pharmaceutical composition according to claim 3, wherein the pharmaceutical composition has the pH of about 5.0 to about 6.0, about 5.2 to about 6.2, about 5.5 to about 6.0, about 5.5 to about 5.8, or about 5.8.
